(19) United States
(12) Patent Application Publication
(10) Pub. No.: US 2004/0171601 A1
(43) Pub. Date: Sep. 2, 2004

(54) Photodynamic and sonodynamic therapy

(76) Inventors: Dai Fukumura, Newton, MA (US); Dennis E.J.G.J. Dolmans, Zeist (NL); Rakesh K. Jain, Boston, MA (US); John S. Hill, Solvang, CA (US)

Correspondence Address: FISH & RICHARDSON PC, 225 FRANKLIN ST, BOSTON, MA, 02110, US

(21) Appl. No.: 10/631,524

(22) Filed: Jul. 31, 2003

Related U.S. Application Data
(63) Continuation-in-part of application No. 10/614,275, filed on Jul. 8, 2003.
(60) Provisional application No. 60/400,334, filed on Jul. 31, 2002.

(51) Int. Cl. A61K 31/555; A61N 1/30

(52) U.S. Cl. 514/185; 514/410; 604/20

(57) ABSTRACT

The present invention pertains to improved methods for the destruction of undesirable tissue using photodynamic therapy (PDT) and/or sonodynamic therapy (SDT). Such methods include the use of fractionated dosing of the photosensitizer or sonosensitizer to ensure that the sensitizer can be found in various compartments of the tissue and appropriate vasculature prior to the application of activating energy such as electromagnetic radiation or ultrasonic energy.

### CROSS-REFERENCE TO RELATED APPLICATIONS

This application is a continuation-in-part of U.S. patent application Ser. No. 10/615,275, filed on Jul. 8, 2003, the contents of which are incorporated herein by reference in its entirety. The present application also claims the benefit of prior U.S. Provisional Application Serial No. 60/400,334 filed on Jul. 31, 2002, the contents of which are incorporated herein by reference in its entirety.

### STATEMENT AS TO FEDERALLY SPONSORED RESEARCH

This invention was made in part with Government support under Grant No. PO1-CA80124 awarded by the National Cancer Institute. The Government, thus, has certain rights in the invention.

### FIELD OF THE INVENTION

The invention relates generally to the field of medicine and, in particular, to treatments for diseases characterized by the presence of vascular and/or neovascular blood vessels and/or hyperproliferative and/or abnormal cells.

### BACKGROUND OF THE INVENTION

Photodynamic Therapy (PDT) is a therapeutic procedure to destroy tissue, preferably pathological tissue, for example, cancer tissue or tissue in blood vessels that occur in disorders characterized by hypervascularization or proliferation of neovascular networks. PDT has also been utilized to enhance wound healing and has also been shown to mediate destruction of avascular tissue, including, for example, hair follicles. In addition, PDT can also be used in a broad spectrum of dermatological diseases such as psoriasis, actinic keratosis, haemangioma, and acne, and has been suggested as a treatment for cardiovascular diseases such as atheromatous plaque and restenosis due to intimal hyperplasia. Pre-clinical and early stage clinical studies have also suggested that PDT may play a role in the induction of immune suppression. Carmeliet, 2003, Nature Medicine, 9:653-660, describes various disorders related to angiogenesis that can be treated by PDT.

Therefore, a desirable biological effect of PDT is the destruction of either or both the cells and surrounding vasculature in a target tissue. Other desirable effects include an enhancement in wound healing response in the absence of tissue and cellular destruction and induction of immune suppression. For example, PDT can be locally administered as a primary therapy for early stage disease, palliation of late stage disease, or as a surgical adjuvant for tumors that show loco-regional spread (Dougherty et al., 1998, J. Nat'l Cancer Inst., 90:889-905). PDT has also been investigated as a palliative treatment for cutaneous recurrence of breast cancer (Khan et al., 1993, Eur. J. Cancer, 12:1686-1690; Mang et al., 1998, Cancer J. Sci. Am., 4:378-384) and has been suggested as a potential therapy for locally invasive breast cancer (Mang, supra; Allison et al.,2001, Cancer, 91:1-8).

In PDT, a photosensitizing agent (termed a “photosensitizer”—see herein for a list of photosensitizers) is delivered to the target tissue and then radiation, most usually light of wavelengths between 250-1000 nm, e.g., 500 to 800 nm, or 600 to 700 nm, is applied to the target tissue. Thus, photosensitizing agents are activated by electromagnetic (EM) radiation. However, photosensitizers can also be activated by acoustic energy, in particular, ultrasonic or sonic energy (see, e.g., Misik and Riesz, 2000, Ann. N.Y. Acad. Sci. 899: 335-348; and U.S. Pat. No. 5,817,048) in a process known as sonosensitization or Sonodynamic Therapy (SDT). In this respect, photosensitizing drugs may also have the potential to act as sonosensitizing drugs, or termed herein as sonosensitizers.

In the process of PDT, activation results in the photochemical transfer of the energy by the photosensitizer molecules to a variety of other molecules in the tissue, resulting in the generation of reactive radical species including, amongst others, singlet oxygen, the superoxide radical, and peroxides and peroxide radicals. For example, previously published methods for administering PDT have described the systemic or local delivery of the photosensitizing agent to the patient, after which the photosensitizing agent is allowed to distribute throughout the target tissue, which is then exposed to EM radiation. The activation of the photosensitizing agent in the tissue leads to, amongst other processes, the generation of radicals and, ultimately, the destruction of the target tissue, or the initiation of biological processes that result in the desired effect upon the target tissue, or, in the case of PDT mediated immune suppression on the local and/or systemic immune response.

The precise mechanism by which sonosensitization occurs has not been fully described; however, some investigators suggest that it is due to the chemical activation of the sensitizing molecule by acoustically generated cavitation bubbles in close proximity to the photosensitizer (Misik and Riesz, supra). In one contemplated use, acoustic energy can be used as an activation pathway. It is believed that by applying an acoustic energy to, for example, peroxyoxalate chemiluminescent systems, oxalic acid derivatives can react with hydrogen peroxide in the presence of a fluorophore to produce a bright emission characteristic of a fluorescing molecule. This reaction can proceed via an energetic key intermediate. The key intermediate is capable of transferring energy to the fluorescing molecule. However, instead of transferring the energy to a fluorescing molecule, the energy is transferred to a sensitizer, thereby, activating the sensitizer by acoustic energy. Accordingly, in a manner similar to PDT, activation of the sensitizer in the technique of SDT results in the generation of radical species including, amongst others, the —H and —OH radicals, as well as the peroxyl and alkoxyl radicals, all of which can mediate biological effects (D. Kessel et al., 1994, Int. J. Radiat. Biol. 66: 221-228; D. Kessel et al., 1995, J Photochem. Photobiology. B.28(3):219-21; and Misik and Riesz, supra).

It is believed that cells within the target field can be destroyed by both apoptotic (Godar, 1999, J. Investig. Dermatol. Symp. Proc., 4:17-23; Oleinick et al., 1998, Radiat. Res., 150(5 Suppl):S146-56) and necrotic pathways (Oleinick et al., 1998, supra). In addition, it has been shown that vasculature and microvasculature in tumors and normal tissues are shut down and destroyed by PDT. The exact mechanisms by which these vascular effects are mediated are unknown, but appear to result in vasoconstriction and/or thrombosis and vascular stasis followed by vessel wall breakdown. The data in the literature suggest that the effects are threshold in nature, in other words, once a critical PDT threshold is reached, vascular destruction results (Dolmans et al., 2002, Cancer Res., 62(7):2151-6; Wang et al., 1997, Br. J. Dermatol., 136:184-189; Liu et al., 1997, Cancer Lett., 111:157-165; Fingar, 1996, J. Clin. Laser. Med. Surg., 14:323-328; Brasseur et al., 1996, Photochem. Photobiol., 64:702-706; van Geel et al., 1996, Br. J. Cancer, 73:288-293; Iliaki et al., 1996, Lasers. Surg. Med., 19:311-323; Schmidt-Erfurth et al., 1994, Ophthalmology, 101:1953-1961; McMahon et al., 1994, Cancer Res., 54:5374-5379; Tsilimbaris et al., 1994, Lasers. Surg. Med., 15:19-31; Fingar et al., 1993, Photochem. Photobiol., 58:393-399; Fingar et al., 1993, Photochem. Photobiol., 58:251-258; Denekamp, 1991, Int. J. Radiat. Biol., 60:401-408; Reed et al., 1989, Radiat. Res., 119:542-552).

With currently available PDT regimens for the treatment of disease, one administers the photosensitizer anywhere from about 15 minutes to about 2 days prior to the application of light to allow the photosensitizer time to accumulate in the target disease tissue and to be cleared from normal tissue. These treatments, however, have met with only limited clinical application. Two concerns in the use of the treatment are safety and effectiveness.

There are possible side effects associated with PDT and SDT. For example, at the target site, these methods have been associated with the development of inflammation with edema and pain, and even necrosis with scarring. With systemically delivered sensitizers formulated in either aqueous or organic solvents, or in liposomal formulations, the side effects can include headaches, nausea, and fever, as well as skin photosensitivity. Moreover, the greater the dosage of sensitizers used, the greater the risk of these, and potentially other, side effects. However, if too little sensitizer is used in the treatment, then there is a greater risk of having only a partial response to treatment or recurrence of disease.

### SUMMARY OF THE INVENTION

The invention is based, in part, on the discovery that when treating a subject by photodynamic therapy (“PDT”), multiple administrations of a photosensitizer (fractionated dosing) given prior to a single dose of activating energy, e.g., light, achieves a more effective and safer PDT treatment than a single administration of a photosensitizer and light treatment. Analogously, similar increases in efficacy and safety are achieved in the process of sonodynamic therapy (“SDT”) using acoustic energy, e.g., ultrasonic energy.

There is more than an additive effect in using fractionated dosing in both PDT and SDT, because the same total amount of photosensitizer or sonosensitizer (generally, “sensitizer”) with fractionated administration shows a significantly improved tumor response to therapy compared to the same total dose given by a single administration. Therefore, one could use less photosensitizer or sonosensitizer in the practice of the present invention to achieve results similar to those achieved using currently available methods, or use similar amounts of photosensitizer or sonosensitizer and achieve results better than those achieved with currently available methods. In either case, the treatments of the present invention are safe, because they expose the patient to relatively low doses of photosensitizer or sonosensitizer and/or fewer repeat administrations of therapy.

In general, the invention features methods for administering PDT and/or SDT (“dynamic therapy” (DT)) to a target tissue, e.g., a tumor, in a subject by a) administering to the subject an effective amount of a first sensitizer at a first time; b) administering to the subject an effective amount of a second sensitizer at a second time after the first time; and, thereafter, c) administering to the target tissue activating energy in an amount and of a wavelength effective to activate the first and second sensitizers, thereby administering DT to the target tissue in the subject.

In these methods, the first and second sensitizers can be the same or different. For example, many known photosensitizers are also activated by acoustic energy, and are thus also sonosensitizers. The first and/or second sensitizers can be photosensitizers, and the activating energy can be electromagnetic radiation, for example, light, e.g., having a wavelength between about 600 and 700 nm. In other embodiments, the first and/or second sensitizers can be sonosensitizers, and the activating energy can be acoustic energy, e.g., ultrasonic energy, e.g., at a frequency of from about 10 kHz to 10 MHz, or the first and second sensitizers are both photosensitizers, and the activating energy is acoustic energy.

In certain embodiments, the first time a sensitizer is administered is sufficiently earlier than the administration of activating energy, e.g., radiation, to enable the first sensitizer to infiltrate into a first tissue compartment in the target tissue. For example, when the target tissue is a tumor, the first tissue compartment can be cells in the tumor. The second time the sensitizer is administered can be sufficiently earlier than the administration of the activating energy to enable the second sensitizer to infiltrate into a second tissue compartment in the target tissue. When the target tissue is a tumor, the second tissue compartment can be vasculature in the tumor.

The method can also include administering to the subject an effective amount of a third sensitizer at a third time, subsequent to the second time, and before administration of radiation. In these methods, the first time a sensitizer is administered can be about 2 to 72 hours prior to administering the activating energy and the second time can be about 15 to 60 minutes prior to administering the activating energy. In specific embodiments, the first time can be about 4 hours prior to administering the activating energy and the second time can be about 15 minutes prior to administering the activating energy.

The first and second sensitizers can be independently selected from the group of: indium-bound pyropheophorbides, pyrrole-derived macrocyclic compounds, porphyrins, chlorins, phthalocyanines, indium chloride methyl pyropheophorbide, naphthalocyanines, porphycenes, porphycyanines, pentaphyrins, sapphyrins, benzochlorins, chlorophylls, azaporphyrins, 5-amino levulinic acid, purpurins, anthracenediones, anthrapyrazoles, aminoanthraquinone, phenoxazine dyes, and derivatives thereof, or others described herein. In specific embodiments, one or both of the first and second sensitizers are independently selected from the group: haematoporphyrin derivatives, benzoporphyrin derivative-monoacid ring A, meta-tetrahydroxyphenylchlorin, 5-aminolevulinic acid, tin ethyl etiopurpurin, boronated protoporphyrin, lutetium texaphyrin, phthalocyanine-4,2-(1-hexyloxyethyl)-2-devinyl pyropheophorbide-alpha, or taporfin sodium. A useful sensitizer is MV6401™ (Indium, chloro[methyl 9-ethenyl-14-ethyl-4,8,13,18-tetramethyl-20-oxo-3-phorbinepropanoato (2-)-N23,N24,N25,N26]-, [SP-4-2-(3S-trans)]-(9CI)).

Effective amounts of the first and second sensitizers can be between about 0.01 mg/kg body weight (BW) and 10.0 mg/kg BW, for example, 5.0, 2.5, 1.0, 0.5, 0.25, 0.1, 0.09, 0.08, 0.07, 0.06, 0.05, 0.04, 0.03, or 0.02 mg/kg of BW. The dose per administration will depend on the total number of administrations for a given total dose.

In certain embodiments, the target tissue is in the subject's eye and the methods can be used to treat an ophthalmologic disorder, such as macular degeneration or choroidal neovascularization. Alternatively, the target tissue can be the subject's skin and the methods can be used to treat a dermatological disorder, such as psoriasis or scleroderma.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIGS. 1A-1F are graphs showing the effect of PDT treatment on tumor growth using a single dose of sensitizing agent.

FIGS. 2A-2F are graphs showing the effect of PDT treatment on tumor growth using one embodiment of the method of the invention.

FIG. 3 is a bar graph of accumulation of photosensitizer in the interstitial compartment of tumors in mice over time.

### DETAILED DESCRIPTION

In general, the new PDT and/or SDT methods (“DT methods”) can be used to treat diseases characterized by the presence of vascular and/or neovascular blood vessels and/or hyperproliferative and/or abnormal cells. Examples of such diseases include cancer, in which case the target tissues include tumor vasculature and cancerous and normal cells. Examples of tumors are gastric cancer, enteric cancer, lung cancer, breast cancer, uterine cancer, esophageal cancer, ovarian cancer, pancreatic cancer, pharyngeal cancer, sarcomas, hepatic cancer, cancer of the urinary bladder, cancer of the upper jaw, cancer of the bile duct, cancer of the tongue, cerebral tumor, skin cancer, malignant goiter, prostatic cancer, cancer of the parotid gland, Hodgkin's disease, multiple myeloma, renal cancer, leukemia, and malignant lymphocytoma. For treatment, the tumor must be penetrable by the activation or activating energy.

The new DT methods are described in further detail in the treatment of tumors, but can also be used in the treatment of diseased and/or inflamed tissues. For example, the new methods are useful for the treatment of ophthalmologic disorders such as age-related macular degeneration, diabetic retinopathy, and choroidal neovascularization; dermatological disorders such as psoriasis and scleroderma; gynecological disorders such as dysfunctional uterine bleeding; urological disorders such as condyloma virus; cardiovascular disorders such as restenosis, intimal hyperplasia, and atherosclerotic plaques; hemangioma; autoimmune diseases such as arthritis; hyperkeratotic diseases; and for hair removal. Normal or diseased tissue on any part of the body can be treated with DT; thus, normal or abnormal conditions of the hematological system, the lymphatic reticuloendothelial system, the nervous system, the endocrine and exocrine system, the skeletomuscular system including bone, connective tissue, cartilage and skeletal muscle, the pulmonary system, the gastrointestinal system including the liver, the reproductive system, the immune system, the cardiovascular system, the urinary system, the ocular system, and the auditory and olfactory systems can be treated using the new methods.

General Methodology

Current methods of using PDT as a treatment include injecting a single dose of a photosensitizer, waiting a sufficient period of time for the photosensitizer to reach its target, and then exposing the target region to light. The new methods are described in Dolmans et al., August 2002, Cancer Res., 62:4289-4294. The fact that photosensitizers are taken up by tumor cells, hyperplastic tissue, hyperproliferating cells, and inflamed tissues has been exploited for decades. In previous studies, the drug accumulates in the target tissue if a sufficient time is provided between drug administration and light activation (Dougherty et al., supra).

There is a growing body of evidence that tumor-host interaction regulates biology and treatment response of tumors (Fukumura et al., 1998, Cell, 94:715-725). Thus, orthotopic tumor models provide clinically relevant information. Prior orthotopic models utilized to study PDT have been limited to prostate, ovarian, and brain cancer. In the experiments described herein, DT was used on an orthotopic breast cancer model. This model has been used to discover that the photosensitizer does not distribute itself evenly within the tissue of the tumor. Thus, shortly after injection into a site, the photosensitizer is found in the vasculature and later, by active or passive methods, the photosensitizer can be found in the tissue of the tumor. Surprisingly, however, at this later time point, minimal photosensitizer is found in the vasculature.

Thus, the invention is based, at least in part, on the recognition that by administering the sensitizer more than once, i.e., at different time points before applying a stimulating or activating energy, such as light, one can ensure that the sensitizer is located throughout the target tissue, e.g., tumor tissue, when the activating energy is applied. For example, as described in the Examples below, when the photosensitizer MV6401 is administered at about 4 hours and again at about 15 minutes prior to the light treatment, the photosensitizer infiltrates into both the vascular and tissue (cellular) compartments of the tumor. The timing of administration of other sensitizers depends on their half-life and mode of action. Many photosensitizers are typically administered far longer prior to light activation than MV6401™. For example, haematoporphyrin derivative (PHOTOFRIN™) and meta-tetrahydroxyphenylchlorin (mTHPC; FOSCAN™) are administered between 24 and 72 hours prior to activation.

The key is to administer a specific sensitizer, e.g., a photosensitizer or sonosensitizer, at a first time sufficiently prior to activation, such that it can infiltrate a first compartment in the target tissue. The same or a different sensitizer is then delivered at a second time, again sufficiently prior to activation such that it can infiltrate into a second compartment in the target tissue.

The invention, however, is not limited to administering two separate sensitizer doses. Instead, the invention relates generally to multiple dosing (fractionated dosing) to capture the sensitizer in various locations throughout the target tissue, e.g., diseased tissue. One can envision, three, four, five or more separate administrations at various time points prior to the application of activating radiation. The number of administrations of sensitizer is limited by convenience and comfort to the patient versus the effectiveness of additional doses. The total drug dose is limited by the maximal tolerated dose, which is dependent on the sensitizer used. However, by fractionating the drug dose, the same effect can be achieved with a lower drug dose, or a higher therapeutic effect can be achieved with the same drug dose.

As described in this exemplary method, the use of fractionated dose DT is superior to single dose DT in that it is both a safer and a more effective treatment for destroying tumor tissue. For example, it is known that high sensitizer doses, e.g., 0.12 mg/kg body weight of MV6401 in the present examples, can be used to induce nearly complete tumor eradication. Such high doses, however, can cause severe tissue damage to surrounding normal tissue, as observed in the mouse tumor model due to the penetration of EM radiation into these surrounding tissues with resultant photosensitizer activation. For example, in mice examined in the exemplary method, hemorrhage of the bladder and destruction of the bowel were observed when the mammary fat pad tumor was treated. Furthermore, while long-term vascular effects can be selective to tumor vessels at low and moderate doses, at high doses, DT can cause similar negative effects on normal blood vessels (Dolmans, et al., 2002, supra). It is, therefore, undesirable to administer sensitizers at high doses using standard administration schemes.

On the other hand, when using fractionated dosing, one does not need to administer high-level doses for any one administration. Instead, the dose would be divided into smaller amounts depending on the number of doses to be administered in the treatment. In other words, instead of one injecting 0.12 mg/kg body weight of sensitizer, one could administer two injections of 0.06 mg/kg body weight of sensitizer, each at a different time point.

Moreover, the cited examples demonstrate that fractionated dosing treatments exhibit greater treatment efficacy than single dose treatments; thus, a smaller amount of sensitizer is needed to produce similar effects. This was a surprising result, because there was no indication as to why this should happen, in view of the fact that both the fractionated dose treatment and the single dose treatment utilized the same total amount of sensitizer and a single activating energy administration. However, from the results of the experiment described below, the fractionated sensitizer dosage regimen appears to provide a synergistic, i.e., more than additive, effect.

There may be several explanations for the profound long-term vascular effects shown with fractionated dosing. First, both luminal and abluminal surfaces of the blood vessel wall contain therapeutic amounts of sensitizer in the tumors exposed to fractionated doses. Thus, DT may effectively attack both endothelial and perivascular cells simultaneously. Second, tumor blood flow is known to be temporally and spatially heterogeneous (Hamberg et al., 1994, Cancer Res. 54: 6032-6036; Jain et al., 1990, Cancer Res. 50: 814s-819s). This effect may lead to a heterogeneous distribution of the sensitizer in the tumor vasculature following a single administration. The new methods of fractionated sensitizer dosing overcome this problem. In addition, fractionated sensitizer dosing permits more homogenous distribution of the sensitizer throughout the tumor vasculature by covering different fractions of temporally perfused vessels.

Fractionation of the light dose, as opposed to the sensitizer, though possible, would require more resources and may be more invasive depending on the application (e.g., peritoneal metastasis). For example, one drawback to multiple treatments of dose-light and varying the timing between treatments to treat the various compartments of the tumor is that it becomes difficult to ensure that the same site on the subject is being irradiated from treatment to treatment. With fractionated dosing, the sensitizer is distributed to the various compartments throughout the tumor and then only a single treatment light is applied to ensure that the proper site is being irradiated. Another drawback to multiple treatments is the invasiveness of some treatments. Some tumors, such as those found in the lungs or ovaries, would require that the means for applying an activating energy to the sensitizer be an invasive one, such as the use of an endoscope.

Examples 2 and 3 described below were conducted using the new method, and show that MV6401, one of a number of useful photosensitizers, induced vascular shutdown and long-term tumor growth delay in an orthotopic breast tumor model in a dose-dependent manner. These results are consistent with studies on mouse dorsal skinfold chamber models, which have shown that thrombus formation is a major cause of long-term vascular shut down (Dolmans et al., 2002, supra). Thus, it is shown herein that the new methods of fractionated dose DT can cause tumor vascular stasis and tumor growth delay in a drug dose-dependent manner, and that a fractionation of the sensitizer is superior to single dosage in mediating these effects.

Combination Therapies Including the New Methods

Fractionated drug dosing has additional uses. In both orthotopic mammary fat pad and dorsal skinfold chamber models, tumor vessel-selective DT may induce only moderate tumor growth control, and tumor regrowth may be proportional to the recovery/regain of blood vessel perfusion resulting in the regrowth of tumors. Tissue perfusion can be recovered by new vessel formation rather than by reperfusion of static vessels. Hypoxia and other stresses induced by DT may up-regulate angiogenic factors such as vascular endothelial growth factor (VEGF) (Ferrario et al., 2000, Cancer Res., 60:4066-4069). Thus, for better long-term tumor control with anti-vascular DT, a combined treatment including DT with anti-angiogenic therapy and/or cytotoxic therapy may be desirable.

Moreover, the therapeutic response of these methods can be improved by fractionation. For example, multiple DT light doses can be given to avoid oxygen depletion during PDT (de Bruijn et al., 1999, Cancer Res., 59:901-904; Hua et al., 1995, Cancer Res., 55:1723-1731). Like chemotherapy, radiation sensitizers and subsequent radiation at one time point have also been fractionated to attack tumor cells that are in different stages of the cell cycle (Kirichenko et al., 1996, Ann. N.Y. Acad. Sci., 803:312-314). Such treatments are designed to eradicate tumors by attacking tumor cells in the different stages of their life cycle. Unlike these treatments, which essentially target a single compartment, e.g., the tumor cells, the new methods attack different compartments of the tumor. The benefit of this new approach is to attack the tumor through different mechanisms of tumor growth, not just stages of cell growth.

Photosensitizers and Sonosensitizers

A variety of molecules can be used as photosensitizers and/or sonosensitizers (herein referred to as “sensitizers”) in the new methods. In certain embodiments, a photosensitizer is a molecule capable of the photochemical conversion of an irradiating energy into radical and cytotoxic species (as described above), which in turn mediates the desired biological effect on target cells and/or blood vessels. In other embodiments, a sonosensitizer is a molecule capable of converting an acoustic energy, such as ultrasound energy, into radical and cytotoxic species. In certain embodiments, an acoustic energy is used to create a key intermediate that is used to transfer energy to activate a photosensitizer. In certain other embodiments, more than one photosensitizer or sonosensitizer can be used in the new methods. The new methods can include the use of a photosensitizer and a sonosensitizer. In still other embodiments, a given molecule can be activated by both electromagnetic energy and acoustic energy, and is thus both a photosensitizer and a sonosensitizer.

In still other embodiments, the photosensitizer is capable of absorbing electromagnetic radiation and transferring that energy by a chemical process to desired target molecules, to biological complexes, and/or cellular or tissue structures. Such an energy transfer may occur in a photochemical process similar to photosynthesis in plants. In other embodiments, a sonosensitizer is capable of absorbing an acoustic energy, such as ultrasonic energy, and transferring that energy to the desired target molecules.

In certain embodiments, sensitizers useful for the described methods include, but are not limited to, the following naturally occurring or synthetic compounds and derivatives thereof: pyrrole derived macrocyclic compounds, porphyrins, chlorins, bacteriochlorins, isobacteriochlorins, phthalocyanines, naphthalocyanines, porphycenes, porphycyanines, pentaphyrins, sapphyrins, benzochlorins, chlorophylls, azaporphyrins, the metabolic porphyrinic precursor 5-amino levulinic acid, PHOTOFRIN®, synthetic diporphyrins and dichlorins, phenyl-substituted tetraphenyl porphyrins (e.g., FOSCAN® picket fence porphyrins), indium chloride methyl pyropheophorbide (MV64013™), 3,1-meso tetrakis (o-propionamido phenyl) porphyrin, verdins, purpurins (e.g., tin and zinc derivatives of octaethylpurpurin (NT2), and etiopurpurin (ET2)), zinc naphthalocyanines, anthracenediones, anthrapyrazoles, aminoanthraquinone, phenoxazine dyes, chlorins (e.g., chlorin e6, and mono-1-aspartyl derivative of chlorin e6), benzoporphyrin derivatives (BPD) (e.g., benzoporphyrin monoacid derivatives, tetracyanoethylene adducts of benzoporphyrin, dimethyl acetylenedicarboxylate adducts of benzoporphyrin, Diels-Adler adducts, and monoacid ring “a” derivative of benzoporphyrin), low density lipoprotein mediated localization parameters similar to those observed with hematoporphyrin derivative (HPD), sulfonated aluminum phthalocyanine (Pc) (sulfonated AlPc, disulfonated (AIPcS₂), tetrasulfonated derivative, sulfonated aluminum naphthalocyanines, chloroaluminum sulfonated phthalocyanine (CASP)), phenothiazine derivatives, chalcogenapyrylium dyes cationic selena and tellurapyrylium derivatives, ring-substituted cationic phthalocyanines, pheophorbide alpha, hydroporphyrins (e.g., chlorins and bacteriochlorins of the tetra(hydroxyphenyl) porphyrin series), phthalocyanines, hematoporphyrin (HP), protoporphyrin, uroporphyrin III, coproporphyrin III, protoporphyrin IX, 5-amino levulinic acid, pyrromethane boron difluorides, indocyanine green, zinc phthalocyanine, dihematoporphyrin, benzoporphyrin derivatives, carotenoporphyrins, hematoporphyrin and porphyrin derivatives, Rose Bengal, bacteriochlorin A, epigallocatechin, epicatechin derivatives, hypocrellin B, urocanic acid, indoleacrylic acid, rhodium complexes, etiobenzochlorins, octaethylbenzochlorins, sulfonated Pc-naphthalocyanine, silicon naphthalocyanines, chloroaluminum sulfonated phthalocyanine, phthalocyanine derivatives, iminium salt benzochlorins, and other iminium salt complexes, Merocyanin 540, Hoechst 33258, and other DNA-binding fluorochromes, psoralens, acridine compounds, suprofen, tiaprofenic acid, non-steroidal anti-inflammatory drugs, methylpheophorbide-a-(hexyl-ether), and other pheophorbides, furocoumarin hydroperoxides, Victoria blue BO, methylene blue, toluidine blue, porphycene compounds described in U.S. Pat. No. 5,179,120, indocyanines, any other photosensitizers noted herein, and any combination of any or all of the above.

The “derivative” or “derivatives” of the sensitizers mentioned above are molecules with functional groups that are attached covalently or non-covalently to the molecule. Examples of the functional groups are: (1) hydrogen; (2) halogen, such as fluoro, chloro, iodo, and bromo; (3) lower alkyl, such as methyl, ethyl, n-propyl, isopropyl, t-butyl, n-pentyl, and the like groups; (4) lower alkoxy, such as methoxy, ethoxy, isopropoxy, n-butoxy, tentoxy, and the like; (5) hydroxy; alkylhydroxy, alkylethers (6) carboxylic acid or acid salts, such as —CH₂COOH, —CH₂COO⁻Na⁺,—CH₂CH₂COOH, —CH₂CH₂COONa, —CH₂CH₂CH(Br)COOH, —CH₂CH₂CH(CH₃)COOH, —CH₂CH(Br)COOH, —CH₂CH(CH₃)COOH, —CH(CI)—CH₂—CH(CH₃)—COOH, —CH₂—CH₂—C(CH₃)₂—COOH, —CH₂—CH₂—C(CH₃)₂—COO⁻K⁺, —CH₂—CH₂—CH₂—CH₂—COOH, C(CH₃)₃—COOH, CH(CI)₂—COOH and the like; (7) carboxylic acid esters, such as —CH₂CH₂COOCH₃, —CH₂CH₂COOCH₂CH₃, —CH₂CH(CH₃)COOCH₂CH₃, —CH₂CH₂CH₂COOCH₂CH₂CH₃, —CH₂CH(CH₃)₂COOCH₂CH₃, and the like; (8) sulfonic acid or acid salts, for example, group I and group II salts, ammonium salts, and organic cation salts such as alkyl and quaternary ammonium salts; (9) sulfonylamides such as substituted and unsubstituted benzene sulfonamides; (10) sulfonic acid esters, such as methyl sulfonate, ethyl sulfonate, cyclohexyl sulfonate, and the like; (11) amino, such as unsubstituted primary amino, methylamino, ethylamino, n-propylamino, isopropylamino, 5-butylamino, secbutylamino, dimethylamino, trimethylamino, diethylamino, triethylamino, di-n-propylamino, methylethylamino, dimethyl-sec-butylamino, 2-aminoethanoxy, ethylenediamino, 2-(N-methylamino) heptyl, cyclohexylamino, benzylamino, phenylethylamino, anilino, -methylanilino, N,N-dimethylanilino, N-methyl-N ethylanilino, 3,5-dibromo-4-anilino, p-toluidino, diphenylamino, 4,4′-dinitrodiphenylamino, and the like; (12) cyano; (13) nitro; (14) a biologically active group; (15) any other substituent that increases the amphiphilic nature of the compounds; or (16) doso- or nido-carborane cages.

The “biologically active group” of the derivative of the sensitizers mentioned above can be any group that selectively promotes the accumulation, elimination, binding rate, or tightness of binding in a particular biological environment. For example, one category of biologically active groups is the substituents derived from sugars, specifically, (1) aldoses such as glyceraldehyde, erythrose, threose, ribose, arabinose, xylose, Iyxose, allose, altrose, glucose, mannose, gulose, idose, galactose, and talose; (2) ketoses such as hydroxyacetone, erythrulose, rebulose, xylulose, psicose, fructose, verbose, and tagatose; (3) pyranoses such as glucopyranose; (4) furanoses such as fructo-furanose; (5) O-acyl derivatives such as penta-O-acetyl-a-glucose; (6) O-methyl derivatives such as methyl a-glucoside, methyl p-glucoside, methyl a-glucopyranoside and methyl-2,3,4,6-tetra-O-methyl glucopyranoside; (7) phenylosazones such as glucose phenylosazone; (8) sugar alcohols such as sorbitol, mannitol, glycerol, and myo-inositol; (9) sugar acids such as gluconic acid, glucaric acid and glucuronic acid, o-gluconolactone, 5-glucuronolactone, ascorbic acid, and dehydroascorbic acid; (10) phosphoric acid esters such as a-glucose 1-phosphoric acid, a-glucose 6-phosphoric acid, a-fructose 1,6-diphosphoric acid, and a-fructose 6-phosphoric acid; (11) deoxy sugars such as 2-deoxy-ribose, rhammose (deoxy-mannose), and fructose (6-deoxy-galactose); (12) amino sugars such as glucosamine and galactosamine; muramic acid and neuraminic acid; (13) disaccharides such as maltose, sucrose and trehalose; (14) trisaccharides such as raffinose (fructose, glucose, galactose) and melezitose (glucose, fructose, glucose); (15) polysaccharides (glycans) such as glucans and mannans; and (16) storage polysaccharides such as a-amylose, amylopectin, dextrins, and dextrans.

Amino acid derivatives are also useful biologically active groups, such as those derived from valine, leucine, isoleucine, threonine, methionine, phenylalanine, tryptophan, alanine, arginine, aspartic acid, cystine, cysteine, glutarnic acid, glycine, histidine, proline, serine, tyrosine, asparagines, and glutamine. Also useful are peptides, particularly those known to have affinity for specific receptors, for example, oxytocin, vasopressin, bradykinin, LHRH, thrombin, and the like.

Other useful biologically active groups are those derived from nucleosides, for example, ribonucleosides such as adenosine, guanosine, cytidine, and uridine; and 2′-deoxyribonucleosides, such as 2′-deoxyadenosine, 2′-deoxyquanosine, 2′-deoxycytidine, and 2′-deoxythymidine.

Another category of biologically active groups that is particularly useful is any ligand that is specific for a particular biological receptor. A “ligand specific for a receptor” is a moiety that binds to a biological receptor, e.g., on a cell surface, and, thus, contains contours and charge patterns that are complementary to those of the biological receptor. Examples of such ligands include: (1) the steroid hormones, such as progesterone, estrogens, androgens, and the adrenal cortical hormones; (2) growth factors, such as epidermal growth factor, nerve growth factor, fibroblast growth factor, and the like; (3) other protein hormones, such as human growth hormone, parathyroid hormone, and the like; (4) neurotransmitters, such as acetylcholine, serotonin, dopamine, and the like; and (5) antibodies. Any analog of these substances that also succeeds in binding to a biological receptor is also included. Particularly useful examples of substituents tending to bind to receptors (and to increase the amphiphilic nature of photosensitizers) include: (1) long chain alcohols, for example, 13 C₁₂H₂₄—OH where —C₁₂H₂₄ is hydrophobic; (2) fatty acids and their salts, such as the sodium salt of the long-chain fatty acid oleic acid; (3) phosphoglycerides, such as phosphatidic acid, phosphatidyl ethanolamine, phosphatidyl choline, phosphatidyl serine, phosphatidyl inositol, phosphatidyl glycerol, phosphatidyl 3′-O-alanyl glycerol, cardiolipin, or phosphatidal choline; (4) sphingolipids, such as sphingomyelin; and (5) glycolipids, such as glycosyidiacylglycerols, cerebrosides, and sulfate esters of cerebrosides or gangliosides.

In certain embodiments, sensitizers useful in the new methods include, but are not limited to, members of the following classes of compounds: porphyrins, chlorins, bacteriochlorins, purpurins, phthalocyanines, naphthalocyanines, texaphyrins, and non-tetrapyrrole photosensitizers. For example, the sensitizer may be, but is not limited to, PHOTOFRIN®, benzoporphyrin derivatives, tin ethyl etiopurpurin (SnET2), sulfonated chloroaluminum phthalocyanines and methylene blue, and any combination of any or all of the above. Examples of oxalate esters that can yield a key intermediate for use with acoustically activated sensitizers can be found in U.S. Pat. Nos. 5,817,048 and 6,233,481.

Any compound, molecule, ion, or atom can be examined for its usefulness for the described methods, for example, by testing it in the mouse model described in the Example Section below. Other animal models known in the art can also be used to test a photosensitizer for its usefulness in the new methods. Such animal models are described in, for example, Bellnier et al., 1995, Photochemistry and Photobiology, 62:896-905; Endrich et al., 1980, Res. Exp. Med., 177:126-134; Tije et al., 1999, Photochem. Photobiol., 69:494-499; Abels et al., 1997, J. Photochem. Photobiol., B40:305-312; Fingar et al., 1992, Cancer Res., 52:4914-4921; Milstone et al., 1998, Microcirculation., 5:153-171; Kuhnle et al., 1998, J. Thorac. Cardiovasc. Surg., 115:937-944; Scalia et al., 1998, Arterioscler. Thromb. Vasc. Biol., 18:1093-1100; Iida et al., 1997, Anesthesiology, 87:75-81; Dalla Via et al., 1999, J. Med. Chem., 42:4405-4413; Baccichetti, et al., 1992, Farmaco., 47:1529-1541; and Roberts et al., 1989, Photochem. Photobiol., 49:431-438. See, also, U.S. Pat. Nos. 5,965,598; 5,952,329; 5,942,534; 5,913,884; 5,866,316; 5,775,339; 5,773,460; 5,637,451; 5,556,992; 5,514,669; 5,506,255; 5,484,778; 5,459,159; 5,446,157; 5,409,900; 5,407,808; 5,389,378; 5,368,841; 5,330,741; 5,314,905; 5,298,502; 5,298,018; 5,286,708; 5,262,401; 5,244,671; 5,238,940; 5,214,036; 5,198,460; 5,190,966; 5,179,120; 5,173,504; 5,171,741; 5,166,197; 5,132,101; 5,064,952; 5,053,423; 5,047,419; and 4,968,715, which describe photosensitizers useful in the new methods.

Dosage of Sensitizers

Sensitizers are used in the disclosed methods at a dosage that facilitates the desired biological effects, for example blood vessel and/or tissue destruction. A useful dosage of a sensitizer for the new methods depends, for example, on a variety of properties of the activating energy (e.g., wavelength, energy, energy density, intensity), the optical properties of the target tissue, and properties of the photosensitizer or sonosensitizer. The upper and lower dosage limits depend on the type of sensitizer used. The sensitizer dosimetry can be determined empirically by those skilled in the art utilizing the methods shown in the examples. One factor in determining the dosage per administration is the number of administrations to be given prior to the administration of activating energy. Thus, in the new methods, the dosage can be lower than typically used with a given sensitizer so that the total of all fractionated doses can be the same or lower than the standard dose for a given sensitizer.

Exemplary total doses for use in the new methods include about 1-2.5 mg/kg body weight (BW) of haematoporphyrin derivative (PHOTOFRIN™) with 50-500 J/cm² activation energy; about 1.2 mg/kg of Tin ethyl etiopurpurin (SnET2; PURLYTIN™, Miravant) with 200 J/cm² activation energy; about 0.6-7.2 mg/kg of Lutetium texaphyrin (LUTEX™) with 150 J/cm² activation energy; about 0.1-0.3 mg/kg of meta-tetrahydroxyphenylchlorin (mTHPC; FOSCAN , Scotia Pharmaceutical, Great Britain) with 8-12 J/cm² activation energy; and about 0.018 mg/kg-0.12 mg/kg of indium chloride methyl pyropheophorbide, which is also known as indium methyl pyropheophorbide, and indium methyl pyropheophorbide-a (the full chemical name is (Indium, chloro[methyl 9-ethenyl-14-ethyl-4,8,13,18-tetramethyl-20-oxo-3-phorbinepropanoato (2-)-N23,N24,N25,N26]-, [SP-4-2-(3S-trans)]-(9CI)); the commercial name is MV6401™, Miravant, Santa Barbara, Calif.) with 5-10 J/cm² activation energy.

Sensitizer Toxicity

In accordance with various embodiments of the present invention, a photosensitizer or sonosensitizer is used at a dosage less than the dosage that would be so toxic to the organism of interest as to render the described methods unfeasible. Toxicological data for many sensitizers are known in the art. See, for example, Ouedraogo et al., 1999, Photochem. Photobiol. 70:123-129; Halkiotis et al., 1999, Mutagenesis 14:193-198; Murrer et al., 1999, Br. J. Cancer 80:744-755; Mandys et al., 1998, Photochem. Photobiol. 47:197-201; Muller etal., 1998, Toxicol. Lett. 102-103:383-387; Waterfield et al., 1997, Immunopharmacol. Immunotoxicol 19:89-103; Munday et al, 1996, Biochim. Biophys. Acta 1311:1-4; Noske et al., 1995, Photochem. Photobiol. 61:494-498; and Lovell et al., 1992, Food Chem. Toxicol 30:155-160.

The toxicity of a photosensitizer or sonosensitizer at any dosage can be determined using the animal model described in detail in the Example Section below. Other animal models are known to the skilled artisan and are discussed in the references previously provided at the end of the Photosensitizer and Sonosensitizer section.

Modes of Formulating and Administering Sensitizers

Sensitizers useful in the described methods can be prepared or formulated for administration in any medium known to the skilled artisan including, but not limited to, tablet, solution, gel, aerosol, dry powder, biomolecular matrix, inhalation. The U.S. Patents at the end of the Photosensitizer and Sonosensitizer section describe the formulation and administration of photosensitizers useful in the described methods.

Sensitizers useful in the new methods can be administered to a subject by any means known to the skilled artisan including, but not limited to, oral, systemic injection (e.g., venous, arterial, lymphatic), local injection (e.g., slow release formulations), hydrogel polymers, inhalation delivery (e.g., dry powder, particulates), electroporation-mediated, iontophoresis or electrophoresis-mediated, microspheres or nanospheres, liposomes, erythrocyte shells, implantable delivery devices, local drug delivery catheter, perivascular delivery, pericardial delivery, eluting stent delivery.

Sensitizers can also be conjugated to targeting agents, such as antibodies directed to specific target tissues (e.g., tumor-associated antigens or vascular antigens, such as the ED-B domain). Ligands directed against receptors that are up-regulated in tumor cells can also be conjugated to sensitizers. For example, low-density lipoprotein (LDL) can be conjugated to sensitizers to be directed at tumor cells that express the LDL receptor, and estrogen can be used to target sensitizers to estrogen receptor expressing cells, such as found in hormone-dependent tumors. Liposomes and immunoliposomes can also be used as targeting agents to carry the sensitizers to specific target tissues.

Activating Energy

Once the fractionated dosage of sensitizer(s) is administered to the subject, the sensitizer(s) must be activated by the proper dosage of electromagnetic (EM) radiation, e.g., light, or acoustic energy, e.g., ultrasound. The power, intensity, and duration of the activating radiation used in the new methods, is calibrated so that it facilitates the desired biological effect(s), such as cellular and/or blood vessel destruction at the selected site in the organism of interest when applied to the chosen type and dose of sensitizer(s). Radiation used in the described methods is preferably calibrated, for example, by choosing the appropriate wavelength, power, power density, energy density, and time of application relative to the times of supply of the photosensitizer(s) to the organism. The wavelength of the radiation can be any wavelength absorbed by the photosensitizer(s), or any other wavelength that mediates the desired biological response in the target tissue.

Some examples of type of photosensitizer, dosage, and activating energy are provided above. For sonic activation, energy in the kilohertz to megahertz range, e.g., one kilohertz to 100 megahertz, can be used. See, also, U.S. Pat. Nos. 6,233,481; 6,013,053; 6,011,563; 5,976,175; 5,971,918; 5,961,543; 5,944,748; 5,910,510; 5,849,027; 5,845,640; 5,835,648; 5,817,048; 5,798,523; 5,797,868; 5,793,781; 5,782,895; 5,707,401; 5,571,152; 5,533,508; 5,489,279; 5,441,531; 5,344,434; 5,219,346; 5,146,917; and 5,054,867, which describe radiation techniques useful in the new DT methods. In particular, U.S. Pat. Nos. 5,817,048 and 6,233,481 describe the use of acoustic energy, such as ultrasonic energy, to activate photosensitizers.

Specific photosensitizers and their activating wavelengths include: MV6401™, 664 nm; PHOTOFRIN™, 630 nm; SnET2, 664 nm; LUTEX™, 732 nm; benzoporphyrin derivative-monoacid ring A (BPD-MA), 689 nm; mTHPC, 652 nm; 5-aminolevulinic acid (5-ALA, LEVULANT™), 635 nm, and boronated protoporphyrin (BOPP), 630 nm. Other useful photosensitizers and their respective activation wavelengths are listed in Dolmans et al., 2003, Nature Reviews, 3:380-387 (Table 1).

In certain embodiments, the wavelength is chosen so that the toxicity to the organism is maintained at a level that does not prohibit the application of the described methods, preferably at a low level, and most preferably at a minimal level. The radiation wavelength used in the new methods is absorbed by the photosensitizer used. In certain embodiments, the radiation wavelength used is such that the absorption coefficient at the chosen wavelength for the photosensitizer used is at least about 5 percent of the highest absorption coefficient for that photosensitizer on the spectrum of electromagnetic radiation of from about 280 nm to about 1700 nm. However, the radiation wavelength may be at least 10, 20, 40, 50, 80, 90, or even 100 percent of the highest absorption coefficient. In other words, the radiation wavelength used in the described methods is such that the absorption coefficient at the chosen wavelength for the photosensitizer used is from about 5 percent to about 100 percent of the highest absorption coefficient for that photosensitizer on the spectrum of electromagnetic radiation of from about 280 nm to about 1700 nm. If more than one photosensitizer is used in the described methods, the above values should apply to at least one of the photosensitizers used, and may apply to all the photosensitizers used.

In certain other embodiments, the wavelength used in the described methods is from about 200 nm to about 2,000 nm, e.g., from about 240 nm to about 1,850 nm, 280 to 1,700 nm, 330 nm to 1,500 mn, 380 nm to 1,250 nm, 330 nm to 1,000 nm, 500 nm to 800 nm, or 600 nm to 700 nm. In certain embodiments, the wavelengths provided above are the wavelengths of the radiation used as it is emitted form the source of radiation used.

In other embodiments, the frequency of the activating ultrasonic energy is from about 10 kHz to 10 MHz, e.g., from about 40 kHz to about 5 M Hz, or from about 100 kHz to about 5 M Hz. In certain embodiments, the frequency and wavelengths provided above are the frequency and wavelengths of the radiation used as it is emitted from the source of radiation used.

The frequency and wavelength of radiation useful for the new methods can be determined using the animal model described in detail in the Examples Section below. Other animal models are known to the skilled artisan and are discussed in the references cited at the end of the Photosensitizer and Sonosensitizer section.

Sources of Activating Energy

In accordance with the invention, any radiation source producing a frequency or wavelength that can activate the photosensitizer used can be employed in the new methods. In certain embodiments, the radiation source used can be a coherent or a non-coherent source including, but not limited to, a laser, a lamp, a light, an optoelectric magnetic device, or a diode, and may include devices that generate ultrasonic energy.

The radiation source must be capable of directing radiation to a site of interest, for example, a laser with optical fiber delivery device, or a fiber optic insert, or a lens used for interstitial or open field light delivery, or diffusers, including those that may improve penetration of the radiation through the skin or a node of a tumor, or an ultra-sonic energy delivery device incorporated into an intra-vascular catheter or interstitial probe or device used to direct ultrasonic energy into the tissue of interest from the surface of the body or surface of tissue. U.S. patents cited at the end of the Activating Energy section describe sources of radiation useful for the described methods.

The usefulness of an energy source can be determined using the mouse model described in detail in the Examples Section below. Other animal models are known to the skilled artisan and are discussed in the references cited at the end of the Photosensitizer and Sonosensitizer section.

### EXAMPLES

The invention is further described in the following examples, which do not limit the scope of the invention described in the claims.

Sensitizer Used in the Examples

The photosensitizing agent, MV6401™ (Miravant Medical Technologies, Santa Barbara, Calif.) is a methyl pyropheophorbide derivative with Indium chelated in the center of the pyropheophorbide macrocycle, as previously reported (Dolmans et al., supra). The molecular weight of MV6401 is 696.9 Dalton. For systemic intravenous administration, the drug was dissolved in a solution of Egg Yolk Phospholipid (EYP) liposomes, which was predominantly composed of cationically charged egg yolk phosphotidyl choline vesicles with an average diameter of 200 nm.

Animal and Tumor Model for the Examples

The experiments were performed in female severe combined immunodeficient (SCID) mice of 8-10 weeks of age. MCaIV murine mammary adenocarcinoma cells, derived from sequential passage (limited to 4 passages) of tumors in these mice were used. A single cell suspension was prepared from minced tumor slurry suspended in a mixture (1:3) of Trypsin 0.25% (Gibco 150-065) and Hanks (Sigma H9269), filtered through SWINEX style filters (Millipore, 13 mm) and centrifuged for 5 minutes. Mice were anesthetized (9 mg ketamine HCl and 0.9 mg xylazine per 100 g body weight, s.c.) and 0.03 ml of the cell suspension was injected into the mammary fat pad inferior to the nipple using a 28 gauge needle, as previously described (Monsky et al., 2002, Clinical Cancer Res., 2002, 8:1008-1013). Care was taken to avoid leakage of cells to subcutaneous space.

Statistical Analysis

All data are expressed as the mean ±SE. The percentage of perfused regions was calculated as 100×number of regions with flow/number of regions examined in each tumor at each time point. Chi-squared tests were performed to compare the proportions. Kaplan-Meier survival analysis was used to compare the survival time between groups. Kaplan-Meier curves were compared using the Logrank test (StatView), and significance was assumed at the 5% confidence level. The animal survival time is defined as the time period from initiation of treatment until exclusion of the animal from the study.

Example 1—Dose-Dependent Tumor Growth Delay

Mice were treated when the tumor reached approximately 14 mm³ (3 mm in diameter). The photosensitizer, MV6401, was injected systemically via tail vein following anesthesia. Mice were positioned in the backs and the tumor area was treated with 5 J/cm² of 664 nm light, delivered from a one Watt diode laser (Type DD2, Miravant Medical Technologies Santa Barbara, Calif.) using an optical fiber with a micro-lens delivery attachment which projected a circular treatment zone of light with even fluence. The light intensity incident on the treatment site was maintained at 50 mW/cm², and to ensure complete treatment of the tumor, the light beam was projected such that there was a 1 mm margin extending beyond the tumor edge. The light dose and light administration protocol remained the same in all experiments performed.

First, the PDT effects mediated by three different doses of MV6401, namely 0.12, 0.06, and 0.03 mg/kg BW (body weight) were examined (Dolmans et al., 2002, supra). The following control groups were also studied: animals that received no drug and no laser treatment, animals that received light alone (up to a maximum dose of 10 J/cm² at a light intensity of 100 mW/cm²), animals that received EYP vehicle alone, and animals that received drug alone (up to a maximum dose of 0.24 mg/kg BW). The interval between drug and light administration was 15 minutes.

Tumor dimensions were determined by caliper measurements every second day following treatment. The volume of each tumor was calculated as p/6×a×b×c (where a is the longitudinal diameter, b is the short diameter and c is the thickness) (Tsuzuki et al., 2001, Lab Invest., 81:1439-1451). When a tumor reached a volume of more than 900 mm³, the mouse was excluded from the study and sacrificed. Surviving mice were monitored up to 60 days post PDT treatment.

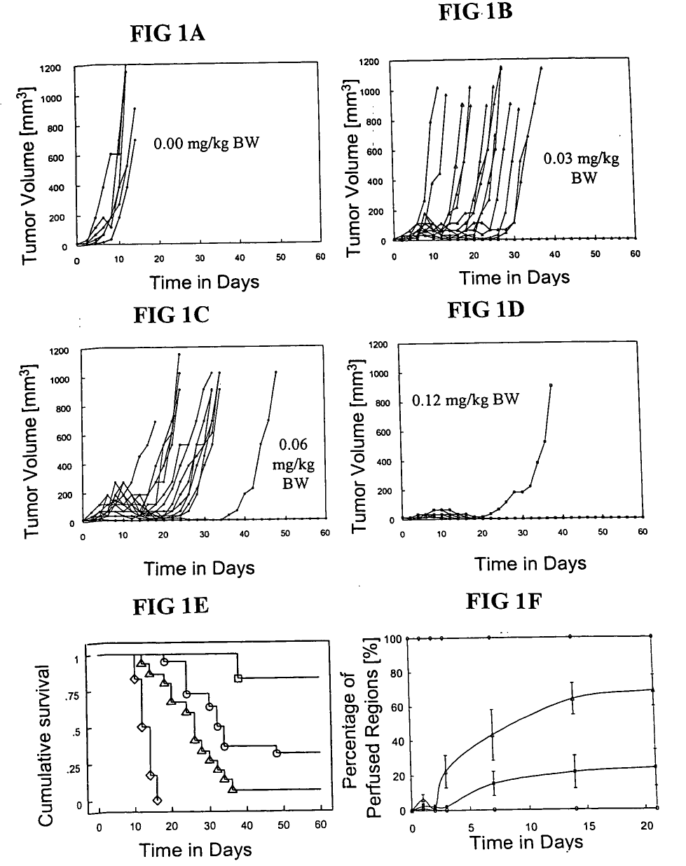

Measurements of the tumors grown in the mammary fat pad showed a drug dose dependent growth delay following PDT with a single dose of MV6401. In FIGS. 1A-1D, each line represents a single animal. In every treatment group, there was a 15-minute interval between drug administration and light treatment (5 J/cm²). Tumor size was measured every two days. FIG. 1A is a graph of the control animals, no treatment (⋄, n=6). FIG. 1B is a graph of the animals treated with 0.03 mg/kg BW MV6401 (Δ, n=15). FIG. 1C is a graph of the animals treated with 0.06 mg/kg BW MV6401 (◯, n=22). FIG. 1D is of the animals treated with 0.12 mg/kg BW MV6401 (□, n=7).

The growth curves show individual tumors and the treatment groups show a significant growth delay compared to the control group. Drug alone and light alone did not affect tumor growth. There was an inverse correlation between drug dose and tumor growth. Thus, PDT with MV6401 induces dose-dependent tumor growth delay.

The survival results are shown in FIG. 1E, which is a graph of a Kaplan-Meier survival curve. Once the tumor reached a volume of 900 mm³, the animal was excluded from the study. Otherwise, the animals were monitored for 60 days: control animals (⋄, n=6), 0.03 mg/kg BW MV6401 group (Δ, n=15), 0.06 mg/kg BW MV6401 group (◯, n=22), and 0.12 mg/kg BW MV6401 group (□, n=7). There was a statistically significant difference between each of the treatment groups (p<0.05, Logrank test). The mean (50%) survival times in the no treatment group, the 0.03 mg/kg BW group and 0.06 mg/kg BW group were 12 days, 24 days, and 32 days, respectively. Treatment with a dose of 0.12 mg/kg BW completely arrested tumor progression except for one tumor in one mouse. However, there was evidence of surrounding normal tissue damage with this high dose. In the underlying tissue (colon, bladder), there was macroscopic and microscopic hemorrhage that did not recover within the experimental period. No mice in the lower dose groups (0.03 or 0.06 mg/kg BW) showed macroscopic evidence of adverse effects on normal tissue.

Intravital microscopy measurements in the mammary fat pad were performed as described previously (Dolmans et al., 2002, supra; Monsky et al., supra; Leunig et al., 1992, Cancer Res., 52: 6553-6560). Briefly, anesthetized animals were injected intravenously with 100 μl of 10 mg/ml FITC-labeled dextran solution (MW, 2,000,000; Sigma Chemical Co., St. Louis, Mo.). Epi-illumination was performed using a 100 W mercury lamp equipped with a fluorescence filter for FITC (excitation: 525-555 nm, emission: 580-635 nm). An intensified charge-coupled device video camera (C2400-88, Hamamatsu Photonics K.K., Hamamatsu, Japan) was used to visualize microvessels in five random areas of each tumor. Before PDT, during PDT and at 1, 2, 3, 7, 14, and 21 days after PDT, the blood vessel perfusion was measured in five random areas in the tumor.

Experiments show that regardless of the drug dose, tumor blood flow stasis was observed in all regions examined during and immediately after PDT. FIG. 1F shows a graph of blood vessel perfusion. The data points show the percentage of regions (5 regions/animal) that exhibited blood flow as determined by intravital microscopy. Data are expressed as mean±SEM. At time=0, PDT was completed. Immediately after PDT, the blood vessel perfusion stopped in all treatment groups. After 2 days there is a significant difference between the treatment groups: control group (⋄, n=5), 0.012 mg/kg BW MV6401 dose group (□, n=5), 0.06 mg/kg BW MV6401 group (◯, n=5), and 0.03 mg/kg BW MV6401 group (Δ, n=10).

Stasis persisted for two days in all treatment groups, and blood flow did not recover in any regions of the tumors in the 0.12 mg/kg BW group up to 21 days following PDT. At time points longer than 2 days post treatment there was resumption of blood flow in tumor vessels treated at the lower doses, as observed by intravital microscopy. The rate of recovery in the 0.06 mg/kg BW group was slower than in the 0.03 mg/kg BW group. Analysis undertaken 3 weeks after treatment showed that 100%, 68%, 24%, and 0% of the regions were perfused in the 0.0, 0.03, 0.06, and 0.12 mg/kg BW treated animals, respectively. Animals that received drug alone or light alone did not exhibit altered blood flow compared to the control animals. Thus, DT with MV6401 induces dose-dependent tumor blood flow stasis.

Example 2—Fractionated Dosing

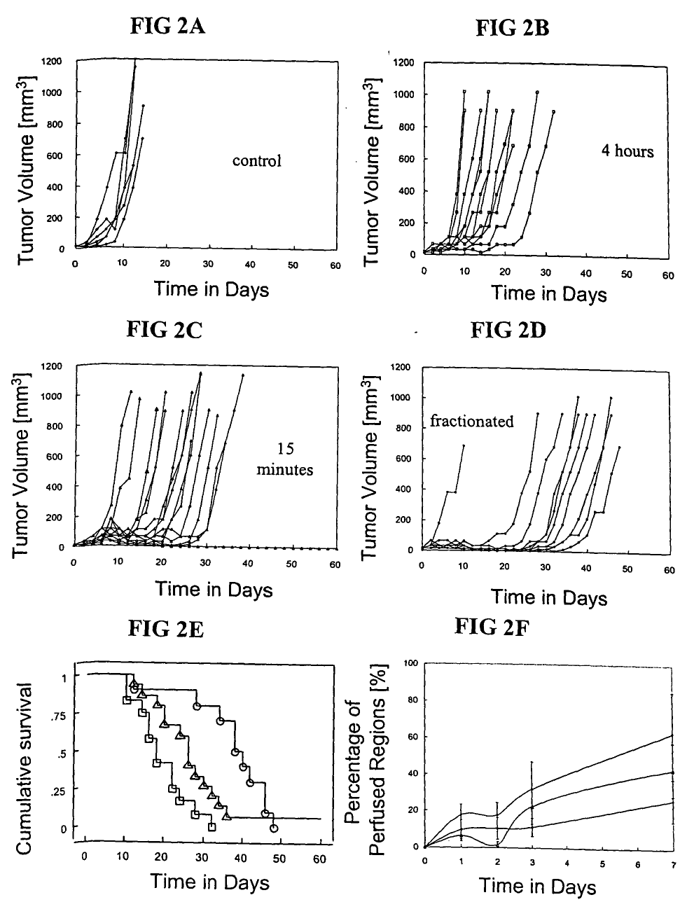

Using the methodology set forth in Example 1, the effects of MV6401 in single dose treatments and in fractionated dose treatments, using a total drug dose of 0.03 mg/kg BW, were examined. Three treatment groups: (i) a four hour group (0.03 mg/kg BW, 4 hours prior to light administration)(FIG. 2B), (ii) a 15 minute group (0.03 mg/kg BW, 15 minutes prior to light administration)(FIG. 2C), and (iii) a fractionated dose group (0.015 mg/kg BW, 4 hours and 0.015 mg/kg BW, 15 minutes prior to a single light administration)(FIG. 2D) were studied. In the last group, the time interval between the two drug doses was 3 hours and 45 minutes.

The total dose of 0.03 mg/kg BW is sub-optimal as a single dose, as shown in FIGS. 1B, but is used to compare effectiveness of fractionated dose treatments over single dose treatments. When the total drug dose of 0.03 mg/kg BW was fractionated into two equal drug doses and the fractions were administered at 4 hours and 15 minutes prior to the light exposure, a significant tumor growth delay was observed (FIG. 2D) compared to single full drug dosing at either 4 hours (FIG. 2B) or 15 minutes (FIG. 2C) prior to light administration. In FIGS. 2A-2D, each line representing a single animal, and in every treatment group the total drug dose (0.03 mg/kg BW MV6401) and the total light dose (5 J/cm²) remained the same.

As before, tumor size was measured every two days. FIG. 2A shows the results of the control animals (⋄, n=6). FIG. 2B shows the results of animals treated with light 4 hours after drug administration (□, n=12). FIG. 2C shows the results of the animals treated with light 15 minutes after drug administration (Δ, n=15). FIG. 2D shows the results of the animals treated with light after fractionated dosing at 4 hours and 15 minutes before the light administration (◯, n=10). With fractionated dosing treatments, the growth of the tumor was delayed for a significantly longer period than single dose treatments.

FIG. 2E shows the Kaplan-Meier survival curve. Again, when the tumor reached a volume of 900 mm³, the animal was excluded from the study; otherwise, the animals were monitored for 60 days: the 15 minutes group (Δ, n=15), the 4 hours group (□, n=12), and the fractionated dose group (◯, n=10). The mean (50%) survival time in the fractionated dose group, the single dose 15 minutes group and the single dose 4 hours group were 38 days, 24 days and 16 days, respectively. Statistical analysis showed that these survival data for the fractionated dose of drug were significantly different (p<0.05, Logrank test) to the data from either of the single dose groups. Because the total dose used (0.03 mg/kg BW) for the single-dose and fractionated dosing regimens was relatively low, a dose of only 0.015 mg/kg BW in a single-dose regimen would likely show less than half the effect of the 0.03 mg/kg dose. Thus, the results would show a greater than additive effect, or synergistic effect, for the fractionated dosing.

In addition, analysis of vascular perfusion of tumors treated in the fractionated dose group, showed that the 15 minutes group and the 4 hours group treatment regimes caused blood flow stasis during and immediately after PDT, see FIG. 2F. (The data points show the percentage of regions (5 regions/animal), which exhibited blood flow as determined by intravital microscopy. Data are expressed as mean±SEM. At time=0, PDT was completed). Immediately after PDT, the blood vessel perfusion stopped in all treatment groups. After 7 days, there was a significant difference between the group that received the fractionated dose (◯, n=5) and the group that received a single drug dose at 4 hours before light treatment (□, n=5). It is noted that tumors in the fractionated dose group showed the most extensive long-term effect on the blood flow with 63%, 43%, and 26% of the regions perfused in the 4-hours group, 15-minutes group, and fractionated dose group, respectively, one week after PDT. Thus, fractionated dose treatments yield better results than single dose treatments.

Example 3—Photosensitizer Localization

Localization of MV6401 in tumors was determined by examining the fluorescence of MV6401 in tissue sections. In brief, the drug and the endothelial cell marker CD31 (PECAM) were visualized back-to-back in serial sections. MV6401 was visualized using epi-fluorescent microscopy. Immuno-fluorescence techniques were used to visualize CD31. Sections were counterstained with DAPI (4′,6-diamidino-2-phenylindole dihydrochloride) to visualize the distribution of drug, blood vessels, and nuclei (Dolmans et al., 2002, supra). In this set of studies, there were 5 groups of mice and their tumors were harvested at different time points, namely: (i) 15 minutes after MV6401 administration, (ii) 4 hours after MV6401 administration, (iii) after the fractionated MV6401 dose administration (4 hours and 15 minutes), (iv) 15 minutes after the EYP administration, and (v) 4 hours after the EYP administration.

Because of the reactivity of Reactive Oxygen Species (ROS) generated by PDT, it is probable that only cells proximal to the area of ROS production will be directly damaged by PDT. Hence, the sites of localization of the photosensitizer in the tumor vasculature and tissue at the time points corresponding to the treatment regimes described above were determined. Images of CD31 staining and DAPI staining and MV6401 fluorescence and DAPI staining in the same regions provide information about localization of MV6401. Images were taken of tissue sections containing EYP, photosensitizer carrier, 15 minutes after the injection; MV6401, 15 minutes after the injection; MV6401, 4 hours after the injection; and MV6401, after fractionated doses (15 minutes and 4 hours).

There was no detectable fluorescence corresponding to the wavelength of emission from MV6401 in cryosections of tumors from animals that received the EYP vehicle alone. Sequential immunohistochemical staining with antibody to CD31 (PECAM) showed that MV6401 co-localized with CD31 positive structures 15 minutes after administration, indicating that MV6401 was confined to the vascular compartment at this time and seemed to be associated with the endothelial cells lining the vessels. When the drug distribution images were superimposed with DAPI-stained nuclear images, MV6401 was observed only in the vascular space and/or associated with the vascular wall. No drug was detected in the surrounding tumor tissue. Similar analysis of tumor sections 4 hours after drug administration showed the drug was mainly localized outside the vascular compartment, with some residual drug associated with the vessel wall. Analysis of the drug distribution of tumors with fractionated drug dose showed that MV6401 was localized both to the interstitial and vascular compartment. Vessel walls, identified as CD31 positive structures, were surrounded by MV6401 from the luminal as well as from the abluminal side.

In another study, intravital fluorescence microscopy was used to determine MV6401 accumulation before, immediately after drug administration, and at 30, 60, 120, 240, or 360 minutes after drug administration in the tumor interstitial tissue, which were devoid of any blood vessels. To determine the optimal time interval between drug and light administration for targeting the tumor cells we quantified the plasma clearance of MV6401 and accumulation of the drug in the interstitial compartment by fluorescent intravital microscopy. For plasma clearance, MV6401 (0.12 mg/kg BW) was intravenously injected and a small amount of arterial blood was collected into micro hematocrit capillary tubes (Fisher Scientific, Pittsburgh, Pa.) before, 3.75, 7.5, 15, 30, 60 minutes after the injection. The capillary tubes were centrifuged and the plasma was transferred to precision rectangle glass capillary tubing with a path length of 0.1 mm (Vitro dynamics, Inc, Rockaway, N.J.). MV6401 fluorescence intensity in the plasma was measured by a photomultiplier (9203B; EMI, Rockaway, N.J.) using an excitation filter (band pass, 390-440 nm), an emission filter (band pass, 665-740 nm), and a dichroic mirror (cutoff frequency, 450 nm) (see Yuan et al., 1994, Cancer Res., 54:3352-3356). Plasma half-life of MV6401 was calculated by curve-fitting plasma pharmacokinetics to an exponential function (n=3), and was 19.5+/−3.1 minutes in SCID mice.

For the interstitial accumulation study, blood vessels in a MCaIV tumor in a dorsal skin fold chamber were visualized using FITC-labeled dextran to exclude blood vessels from regions of interest (ROI) in the interstitial compartment. A specially designed motorized microscope stage (OPTISCAN™ Model ES102/IS102 XY Stage System; Prior Scientific, Inc., Rockland, Mass.) was used to return to each ROI repeatedly before and after the drug administration. 0.12 mg/kg BW MV6401 was injected into each mouse and the fluorescence of the drug was visualized using the same filter set as described above. Only ROI (50 μm in diameter) were illuminated using a minimum size diaphragm in the excitation light path. The fluorescence was measured before, directly after, and at 30, 60, 120, 240, and 360 minutes after MV6401 administration, and the intensity was analyzed off line using NIH Image (version 1.62). Background auto-fluorescence of each ROI obtained before the drug administration was subtracted from subsequent measurements.

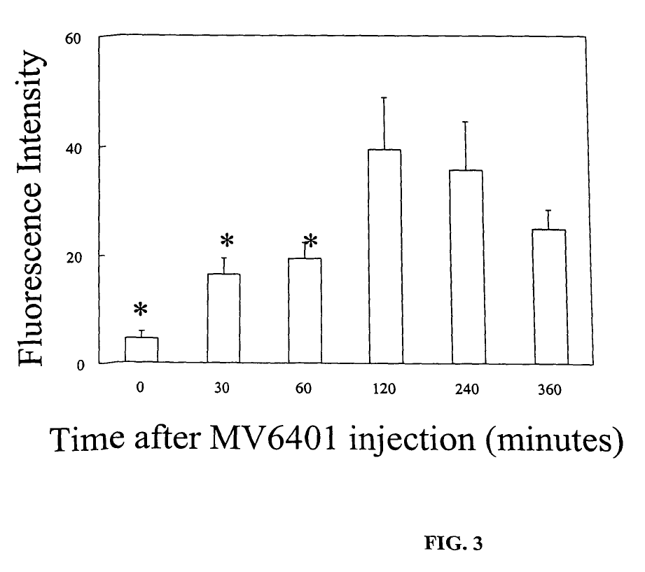

As shown in FIG. 3, at between 60and 120 minutes a significant increase in MV6401 accumulation in the interstitium was observed. However, at 120, 240, and 360 minutes no statistically significant change in photosensitizer signal was observed. The degree, duration, and peak time of the drug accumulation were heterogeneous within the tumor as well as among tumors. Thus, 2 to 6 hours after the injection of MV6401 would be the window for targeting the tumor interstitium. The 4 hours time point was chosen in other experiments to cover most of the areas with relatively high accumulation of the drug.

These experiments show that photosensitizers, such as MV6401, are initially located in the vasculature. However, as time progresses, the photosensitizer diffuses out of the vasculature and into the tumor tissue, leaving only a residual amount of photosensitizer in the vessel walls. Therefore, based on this finding, to effectively destroy the tumorous tissue, one should use multiple dosing of photosensitizer at different time points prior to activation with light to ensure that the photosensitizers have time to enter multiple compartments of the tumor tissue. In this manner, a single PDT treatment can attack the tumorous tissue on many fronts, e.g., the tissue as well as the vasculature.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for administering dynamic therapy (DT) to a target tissue in a subject, the method comprising:
a) administering to the subject an effective amount of a first sensitizer at a first time;
b) administering to the subject an effective amount of a second sensitizer at a second time after the first time; and, thereafter,
c) administering to the target tissue activating energy in an amount and of a wavelength effective to activate the first and second sensitizers, thereby administering DT to the target tissue in the subject.

2. The method of claim 1, wherein the first and second sensitizers are the same.

3. The method of claim 1, wherein the first and second sensitizers are different.

4. The method of claim 1, wherein the first and second sensitizers are photosensitizers, and the activating energy is electromagnetic radiation.

5. The method of claim 1, wherein the first and second sensitizers are sonosensitizers, and the activating energy is acoustic energy.

6. The method of claim 1, wherein the first and second sensitizers are photosensitizers, and the activating energy is acoustic energy.

7. The method of claim 1, wherein the first time is sufficiently earlier than the administration of activating energy to enable the first sensitizer to infiltrate into a first tissue compartment in the target tissue.

8. The method of claim 7, wherein the target tissue is a tumor, and the first tissue compartment is cells in the tumor.

9. The method of claim 1, wherein the second time is sufficiently earlier than the administration of activating energy to enable the second sensitizer to infiltrate into a second tissue compartment in the target tissue.

10. The method of claim 9, wherein the target tissue is a tumor, and the second tissue compartment is vasculature in the tumor.

11. The method of claim 4, wherein the electromagnetic radiation is light.

12. The method of claim 11, wherein the light has a wavelength between about 600 and 700 nm.

13. The method of claim 1, further comprising administering to the subject an effective amount of a third sensitizer at a third time, subsequent to the second time, and before administration of radiation.

14. The method of claim 1, wherein the first time is about 2 to 72 hours prior to administering the activating energy and the second time is about 15 to 60 minutes prior to administering the activating energy.

15. The method of claim 1, wherein the first time is about 4 hours prior to administering the activating energy and the second time is about 15 minutes prior to administering the activating energy.

16. The method of claim 1, wherein the first and second sensitizers are the same or different and are independently selected from the group consisting of: indium-bound pyropheophorbides, pyrrole-derived macrocyclic compounds, porphyrins, chlorins, phthalocyanines, indium chloride methyl pyropheophorbide, naphthalocyanines, porphycenes, porphycyanines, pentaphyrins, sapphyrins, benzochlorins, chlorophylls, azaporphyrins, 5-amino levulinic acid, purpurins, anthracenediones, anthrapyrazoles, aminoanthraquinone, phenoxazine dyes, and derivatives thereof.

17. The method of claim 1, wherein one or both of the first and second sensitizers are independently selected from the group consisting of haematoporphyrin derivatives, benzoporphyrin derivative-monoacid ring A, meta-tetrahydroxyphenylchlorin, 5-aminolevulinic acid, tin ethyl etiopurpurin, boronated protoporphyrin, lutetium texaphyrin, phthalocyanine-4,2-(1-hexyloxyethyl)-2-devinyl pyropheophorbide-alpha, or taporfin sodium.

18. The method of claim 1, wherein one or both of the first and second sensitizers are MV6401™ (Indium, chloro[methyl 9-ethenyl-14-ethyl-4,8,13,18-tetramethyl-20-oxo-3-phorbinepropanoato (2-)-N23,N24,N25,N26]-, [SP-4-2-(3S-trans)]-(9CI))

19. The method of claim 1, wherein an effective amount of the first and second sensitizers is between about 0.01 mg/kg body weight and 10.0 mg/kg body weight.

20. The method of claim 1, wherein the target tissue is a tumor.

21. The method of claim 20, wherein the tumor is a gastric cancer, enteric cancer, lung cancer, breast cancer, uterine cancer, esophageal cancer, ovarian cancer, pancreatic cancer, pharyngeal cancer, sarcomas, hepatic cancer, cancer of the urinary bladder, cancer of the upper jaw, cancer of the bile duct, cancer of the tongue, cerebral tumor, skin cancer, malignant goiter, prostatic cancer, cancer of the parotid gland, Hodgkin's disease, multiple myeloma, renal cancer, leukemia, or malignant lymphocytoma.

22. The method of claim 1, wherein the target tissue is in the subject's eye and the method is used to treat an ophthalmologic disorder.

23. The method of claim 22, wherein the ophthalmologic disorder is macular degeneration or choroidal neovascularization.

24. The method of claim 1, wherein the target tissue is the subject's skin and the method is used to treat a dermatological disorder.

25. The method of claim 24, wherein the dermatological disorder is psoriasis or scleroderma.
